(19) United States
(12) Patent Application Publication
(10) Pub. No.: US 2019/0124940 A1
(43) Pub. Date: May 2, 2019

(54) MANUFACTURE OF CHEESE

(71) Applicant: CHR. HANSEN A/S, Hoersholm (DK)

(72) Inventors: Tatjana VAN DEN TEMPEL, Klampenborg (DK); Thomas JANZEN, Broenshoej (DK); Morten CARLSON, Helsinge (DK)

(73) Assignee: CHR. HANSEN A/S, Hoersholm (DK)

(21) Appl. No.: 15/991,623

(22) Filed: May 29, 2018

Related U.S. Application Data
(63) Continuation of application No. 14/129,158, filed on Dec. 24, 2013, now abandoned.

(30) Foreign Application Priority Data
Jun. 24, 2011 (DK) PA 2011 00477
Jul. 29, 2011 (DK) PA 2011-00577

(51) Int. Cl. A23C 19/032; C12R 1/46; A23C 19/076

(52) U.S. Cl. CPC A23C 19/0323; C12R 1/46; A23Y 2240/75; A23Y 2240/41; A23C 19/076

(57) ABSTRACT

The present invention relates to the manufacture of cheese, especially cottage cheese, by fermenting milk with lactic acid bacteria.

### FIELD OF INVENTION

The present invention relates to the manufacture of cheese, especially cottage cheese, by fermenting milk with lactic acid bacteria.

### BACKGROUND OF INVENTION

Lactic acid bacteria (LAB) are intensively used in the dairy industry for making various animal milk fermented products, e.g. cheese, especially fresh cheese such as cottage cheese. Cottage cheese accounts for approx. 700,000 tons of the world's 18.2 million tons of cheese consumed in 2008. In North America cottage cheese makes up approx. 12% of all cheese. Normally, cottage cheese cultures comprise homofermentative Lactococcus strains such as e.g. Lactococcus lactis strains.

As technological background prior art that describes known methods for making cottage cheese as such may herein be mentioned: U.S. Pat. No. 3,298,836 (published 1967); WO91/00690A1 and the article “Gold Spot Dairy boost cottage cheese sales”, Dairy and ice Cream Field, vol. 156, no. 6. 1973, pages 16-47).

Relatively recently (within the last 3-5 years) Streptococcus thermophilus (ST) has been added to cottage cheese cultures. Addition of S. thermophilus may result in a shorter fermentation time (e.g. shortened to around 4-5 hours). However, S. thermophilus strains are generally capable of expressing the enzyme urease (EC 3.5.1.5), which is an enzyme that catalyzes the hydrolysis of urea into carbon dioxide (CO₂) and ammonia (NH₃). Milk comprises urea. Accordingly, due to the production of the base NH₃ by S. thermophilus there may be a temporary decrease in acidification speed during the fermentation of milk. In relation to the problem of NH₃-induced temporary decrease in acidification speed U.S. Pat. No. 6,962,721B1 (Texel, F R) describes that by using S. thermophilus strains which e.g. are not producing active urease enzyme (so-called “ur(−) strains”) one may get an improved acidification kinetic profile.

Nevertheless, there is a need in the art to provide further improved methods for the manufacture of cottage cheese.

It has now been surprisingly found that during the production of cottage cheese the yield can be significantly improved when using lactic acid bacteria strains that produce capsular-polysaccharides (CPS). Also, the use of CPS-producing bacteria strains was found to improve the physical properties of fermented dairy products.

### SUMMARY OF INVENTION

In it broadest aspect, the present invention relates to a novel method for manufacture of cheese, which comprises the following steps:
a) inoculating a milk substrate with lactic acid bacteria belonging to a strain which is able to produce a capsular polysaccharide (CPS);
b) fermenting the milk with the bacteria; and
c) separating the cheese mass or curd from the whey.

The invention also relates to a novel cheese, such as cottage cheese, obtained by a method of the invention. The cheese can be distinguished from previously known cheeses, as demonstrated e.g. in the example section below (“Conclusion”). Specifically, the overall mouth feel of the cheese is significantly improved in cheese that has been manufactured by the methods described herein. Further, less residual whey is present in the cheese obtained by the methods of the present invention.

Further, the invention relates to the novel use of CPS-producing bacteria belonging to the species Streptococcus thermophilus or Lactococcus lactis in a process for producing cheese, such as cottage cheese, especially the use of a lactic acid bacteria belonging to a strain selected from the group consisting of strains that were deposited with the Leibniz Institute DSMZ—German Collection of Microorganisms and Cell Cultures under accession numbers: DSM24650, DSM24648, DSM24649, DSM24655, DSM24654, DSM21421, DSM25012, and mutants and variants of any of these.

Finally, the invention relates to a novel lactic acid bacterial strain selected from the group consisting of strains that were deposited with the Leibniz Institute DSMZ—German Collection of Microorganisms and Cell Cultures under accession numbers; DSM24650, DSM24648, DSM24649, DSM24655, DSM24654, DSM21421, DSM25012, and mutants and variants of any of these.

### DETAILED DISCLOSURE

In a first aspect, the present invention relates to a method for manufacture of cheese, which comprises the following steps:
a) inoculating a milk substrate with lactic acid bacteria belonging to a strain which is able to produce a CPS (capsular polysaccharide);
b) fermenting the milk with the bacteria; and
c) separating the cheese mass or curd from the whey.

It is contemplated that this method will result in a higher yield of cheese, compared to an identical method, but wherein the strain is not able to produce a CPS. In a particularly preferred embodiment of the invention, the CPS-producing lactic acid bacteria used in the above method belong to a strain which is urease negative, or substantially urease negative.

In another aspect, the invention relates to a method for the manufacture of cheese, which comprises the following steps:
a) inoculating a milk substrate with lactic acid bacteria belonging to a strain which is urease negative, or substantially urease negative;
b) fermenting the milk with the bacteria; and
c) separating the cheese mass or curd from the whey.

It is contemplated that this method will result in a higher yield of cheese, compared to an identical method, but wherein the strain is urease positive.

The above methods will result in a yield that is preferably at least about 1%, about 2%, about 3%, about 4%, about 5%, about 6%, about 7%, about 8%, about 9%, about 10% or more higher than the yield when the same method is carried out with lactic acid bacteria that are not able to produce a CPS and/or that are urease-negative. Preferably, the above methods will result in a yield that is between 0.1 and 20%, between 0.5 and 15% or between 1 and 10% higher than the yield when the same method is carried out with lactic acid bacteria that are not able to produce a CPS and/or that are urease negative.

The lactic acid bacteria strain is preferably a member of the order “Lactobacillales” or “Actinomycetales” which include Lactococcus spp., Streptococcus spp Lactobacillus spp., Leuconostoc spp., Pseudoleuconostoc spp., Pedlococcus spp., Brevibacterium spp., Enterococcus spp. and Propionibacterium spp. In one embodiment of the method of the invention, the strain is a strain of a Lactococcus species, preferably Lactococcus lactis. In another embodiment, the strain is a strain of a Streptococcus species, preferably Streptococcus thermophilus. In a preferred embodiment the lactic acid bacteria strain is a urease negative (Ur−) Streptococcus thermophilus strain.

The skilled person knows techniques that allow the identification of Streptococcus thermophilus strains that are urease negative (Ur−). For example, the strains may be tested biochemically for urease activity under suitable conditions. Further, it is possible to assess whether a strain can be regarded as urease negative by testing for the presence of a functional urease gene or urease protein. The presence of a functional urease protein can be detected, e.g. in an assay that measures the production of dioxide CO₂ and ammonia NH₃ from urea.

The urease negative lactic acid bacteria used in step (a) of the above method are preferably CPS-producing bacteria. As used herein, the term “CPS-producing” lactic acid bacteria is to be understood as comprising any lactic acid bacterium that is capable of producing a capsular polysaccharide, i.e. a polysaccharide which encloses the bacterial cell to form a capsule. Methods for the detection whether a bacterial strain is a CPS-producing strain are well known in the art. Any suitable method for the detection of saccharides or glycoproteins may be also used to determine whether a lactic acid bacterium is able to produce a CPS. For example, the CPS produced by a lactic add bacterium may be detected by CPS-specific antibodies. CPS-producing lactic add bacteria strains can be isolated, e.g. by testing for their hydrophilicity. For example, the distribution of bacterial cells in an organic phase can be compared to their distribution in an aqueous phase. The more cells remain in the aqueous phase, the more hydrophilic the cell surface is, which indicates that a hydrophilic capsular cell envelope is present that is responsible for actively binding water. Without wishing to be bound by theory, it is assumed that it is the water-binding activity of the CPS that is responsible for the increased yield when using CPS-producing lactic add bacteria strains in a method for making cheese.

In a further embodiment, the strain is selected from the group consisting of strains that were deposited with the Leibniz Institute DSMZ—German Collection of Microorganisms and Cell Cultures under accession numbers DSM24650, DSM24648, DSM24649, DSM24655, DSM24654, DSM21421, DSM25012, and mutants and variants of any of these. Specifically, the lactic acid bacteria strain may be selected from the group of Lactococcus lactis subsp. lactis strains that were deposited with the Leibniz institute DSMZ—German Collection of Microorganisms and Cell Cultures under accession numbers DSM24650 and DSM24648, and mutants and variants of any of these. Further, the lactic acid bacteria strain may be selected from the group of Lactococcus lactis subsp, cremoris strains that were deposited with the Leibniz Institute DSMZ—German Collection of Microorganisms and Cell Cultures under accession numbers DSM24649 and DSM21421, and mutants and variants of any of these. In yet another embodiment, the lactic acid bacteria strain may be selected from the group of Streptococcus thermophilus strains that were deposited with the Leibniz Institute DSMZ—German Collection of Microorganisms and Cell Cultures under accession numbers DSM24655 and DSM24654, and mutants and variants of any of these.

In a preferred embodiment, two lactic acid bacteria strains are used in step (a) in the method of the present invention at the same time. For example, two different strains of Streptococcus thermophilus, two different strains of Lactococcus lactis subsp. lactis, two different strains of Lactococcus lactis subsp. cremoris, or two different strains selected from any of the three species may be used at the same time. It is particularly preferred to use two Streptococcus thermophilus strains, e.g. selected from the group of DSM24655, DSM24654 and DSM24653 and mutants and variants thereof, at the same time in the methods of the present invention. It is further preferred to use two Lactococcus lactis strains, e.g. selected from the group consisting of DSM24650, DSM24648, DSM24649, DSM21421 and DSM21404 and mutants and variants thereof, at the same time in the methods of the present invention. It is particularly preferred to use Lactococcus lactis DSM21404 in combination with another Lactococcus lactis strain, selected from the group consisting of DSM24650, DSM24648, DSM24649 and DSM21421 and mutants and variants thereof, at the same time in the methods of the invention. It is further preferred to use Streptococcus thermophilus DSM24653 in combination with another Streptococcus thermophilus strain, selected from the group consisting of DSM24655 and DSM24654 and mutants and variants thereof, at the same time in the methods of the invention.

The “milk substrate” of step (a) may be any raw and/or processed milk material as described hereinafter. The milk substrate may be inoculated with about 10⁴ to 10¹³ cfu/ml (i.e. cell- or colony-forming units per milliliter), preferably about 10⁶ to 10¹² cfu/ml, or more preferably about 10⁸ to 10¹² cfu/ml, of the bacteria.

The method of the invention may comprise a further step of adding a coagulant, such as rennet, rennin, a protease, and/or chymosin, before, during or after step (a). As described hereinafter, “coagulant” refers to any kind of milk clotting agent. In a preferred embodiment, the coagulant added before, during or after step (a) is bovine chymosin.

In step (b), the milk substrate is fermented with the bacteria as defined hereinafter. A relatively long fermentation period is used in the present methods for the manufacture of cheese to allow a pH drop from around pH 6.6, i.e. the pH of milk, to around 4.65. The fermentation time in step (b) may be in the range from 3 to 10 hours, preferably about 7, about 8, about 9 or about 10 hours. Generally, the fermentation time in step (b) will be selected such that it is sufficiently long to allow a pH drop to about 4.65.

In step (c) the cheese mass or curd is separated from the whey. As defined hereinafter, a “cheese” is a product prepared by contacting milk with a coagulant and draining the resultant curd. In a preferred embodiment of the method, the cheese is fresh cheese, such as cottage cheese. Cottage cheese may be seen as a cheese curd product with a mild flavor. It is normally drained but not pressed so some whey may remain and the curd may remain loose. Different types of cottage cheese are made from milk with different fat levels.

It is envisaged that any milk substrate can be used in the method of the invention, but it is presently preferred that the milk substrate is milk, such as cow's milk.

In the method of the invention, step (c) may include one or more of the steps selected from the group consisting of:
i) cutting the coagulum at a pH between 4.6 and 4.7; and
ii) scalding (heating) of the curd after cutting.

In one embodiment, step comprises cutting the coagulum at a pH between 4.0 and 5.5, preferably, between 4.3 and 5.0, more preferably between 4.6 and 4.7. The coagulum is preferably cut into small cubes. For this purpose, a cheese wire or frame cutter can be conveniently used. Preferably, the coagulum is cut into cubes having a site of about 10 mm.

In a further embodiment, step (c) comprises scalding or heating of the curd after cutting. In this step, the curd is normally heated to a temperature between about 45-60° C., more preferably 50-55° C., most preferably 56-57° C., and held at that temperature for about 1-2 hours.

The resulting cheese, curd or cheese curd may be salted, pressed, packaged, added further ingredients, such as cream, food colorants, aromas, or otherwise processed. It is known in the art how to select the appropriate additives and/or processing means to arrive at the desired product. It is preferred that the cheese curd, cheese or curd obtained by the present methods is salted, pressed and packaged. Means for pressing and packaging are known in the art.

An aspect of the present invention relates to curd obtainable, preferably obtained, by the method of the invention, including the processed curd.

In another aspect, the invention relates to cheese, such as cottage cheese, obtained by a method of the present invention.

In yet another aspect, the invention relates to the use of CPS-producing bacteria belonging to the species Streptococcus thermophilus or Lactococcus lactis in a process for producing cheese, such as cottage cheese. In a preferred embodiment, the invention relates to the use of a lactic acid bacteria belonging to a strain selected from the group consisting of strains that were deposited with the Leibniz Institute DSMZ—German Collection of Microorganisms and Cell Cultures under accession numbers: DSM24650, DSM24648, DSM24649, DSM24655, DSM24654, DSM21421, DSM25012, and mutants and variants of any of these in a process for producing cheese, such as cottage cheese.

In a final aspect, the present invention relates to a lactic acid bacterial strain selected from the group consisting of strains that were deposited with the Leibniz Institute DSMZ—German Collection of Microorganisms and Cell Cultures under accession numbers: DSM24650, DSM24648, DSM24649, DSM24655, DSM24654, DSM21421, DSM25012, and mutants and variants of any of these.

In interesting embodiments of this final aspect, the invention relates to a lactic acid bacterial strain selected from the group consisting of strains that were deposited with the Leibniz Institute DSMZ—German Collection of Microorganisms and Cell Cultures under accession numbers: DSM24650, DSM24648, DSM24649, DSM24655, DSM24654, DSM21421, DSM 25012, and mutants of any of these. All of these strains are urease negative and moreover produce a CPS.

A mutant of the present invention may be any mutant which is (substantially) functionally equivalent to the mother strain, e.g. has substantially the same, or improved, properties as the mother strain, for example regarding yield. Alternatively, a mutant of the present invention may be any mutant, wherein the genome of said mutant is at least 99% homologous to the genome of the mother strain. Additionally, a mutant of the present invention may be any mutant wherein less than 1%, such as less than 0.1%, of the nucleotides in the bacterial genome of the mutant have been shifted with another nucleotide, or deleted, compared to the mother strain. Preferably less than 1%, and more preferably less than 0.1%, of the nucleotides in the bacterial genome of the mutant have been replaced by another nucleotide or been deleted in comparison with the genome of the mother strain. By “mother strain” in the present context is meant a strain of the invention, such as a strain selected from the group consisting of strains that were deposited with the Leibniz institute DSMZ—German Collection of Microorganisms and Cell Cultures under accession numbers DSM24650, DSM24648, DSM24649, DSM24655, DSM24654, DSM21421, and DSM 25012.

It should of course be understood that any of the mutants of the invention can be used in the method of the invention.

### DEFINITIONS

In the present context, the term “cheese” refers to a product prepared by contacting milk, optionally acidified milk, such as milk that is acidified e.g. by means of a lactic acid bacterial culture, with a coagulant, and draining the resultant curd. Cheeses and their preparation are described in e.g. “Cheese and Fermented Milk Foods”, by Frank V. Kosikowski.

The term “cottage cheese” includes cheeses or cottage cheeses prepared by any known manufacturing procedure, such as those for instance that are described in the references mentioned herein.

In the present context, the term “coagulant” refers to refers to any kind of milk clotting agent, such as a native enzyme derived from microbial, vegetable or animal tissue sources or a milk clotting enzyme provided as a gene product of recombinant cells expressing a milk clotting enzyme of animal or microbial origin. The term includes bovine chymosin purified from abornasum tissue or made by fermentation (e.g. CHY-MAX (R) or CHY-MAX (R) M).

As used herein, the term “lactic acid bacterium” designates a gram-positive, microaerophilic or anaerobic bacterium, which ferments sugars with the production of acids, such as lactic acid, which is the predominantly produced acid, acetic acid and propionic acid. The industrially most useful lactic acid bacteria are found within the order “Lactobacillales” and “Actinomycetales” which include Lactococcus spp., Streptococcus spp., Lactobacillus spp., Leuconostoc spp., Pseudoleuconostoc spp., Pediococcus spp., Brevibacterium spp., Enterococcus spp. and Propionibacterium spp.

In the present context, the term “milk substrate” may be any raw and/or processed milk material that can be subjected to fermentation according to the method of the invention. Thus, useful milk substrates include, but are not limited to, solutions/suspensions of any milk or milk like products comprising protein, such as whole or low fat milk, skim milk, buttermilk, reconstituted milk powder, condensed milk, dried milk, whey, whey permeate, lactose, mother liquid from crystallization of lactose, whey protein concentrate, or cream. Obviously, the milk substrate may originate from any mammal, e.g. being substantially pure mammalian milk, or reconstituted milk powder. Preferably, at least part of the protein in the milk substrate is proteins naturally occurring in milk, such as casein or whey protein. However, part of the protein may be proteins which are not naturally occurring in milk. Prior to fermentation, the milk substrate may be homogenized and pasteurized according to methods known in the art.

The term “milk” is to be understood as the lacteal secretion obtained by milking any mammal, such as cows, sheep, goats, buffaloes or camels. In a preferred embodiment, the milk is cow's milk. The term milk also comprises non-animal milks, such as soy milk. Optionally, the milk is acidified, e.g. by addition of an acid (such as citric, acetic or lactic acid), or mixed, e.g. with water. The milk may be raw or processed, e.g. by filtering, sterilizing, pasteurizing, homogenizing, etc., or it may be reconstituted dried milk. An important example of “bovine milk” according to the present invention is pasteurized cow's milk. It is understood that the milk may be acidified, mixed or processed before, during and/or after the inoculation with bacteria.

“Homogenizing” as used herein means intensive mixing to obtain a soluble suspension or emulsion. If homogenization is performed prior to fermentation, it may be performed so as to break up the milk fat into smaller sizes so that it no longer separates from the milk. This may be accomplished by forcing the milk at high pressure through small orifices.

“Pasteurizing” as used herein means treatment of the milk substrate to reduce or eliminate the presence of live organisms, such as microorganisms. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow.

“Fermentation” in the methods of the present invention means the conversion of carbohydrates into alcohols or acids through the action of a microorganism. Preferably, fermentation in the methods of the invention comprises conversion of lactose to lactic acid.

Fermentation processes to be used in production of fermented milk products are well known and the person of skill in the art will know how to select suitable process conditions, such as temperature, oxygen, amount and characteristics of microorganism(s) and process time. Obviously, fermentation conditions are selected so as to support the achievement of the present invention, i.e. to obtain a fermented milk product.

Lactic acid bacteria, including bacteria of the species Lactococcus lactis and Streptococcus thermophilus, are normally supplied to the dairy industry either as frozen or freeze-dried cultures for bulk starter propagation or as so-called “Direct Vat Set” (DVS) cultures, intended for direct inoculation into a fermentation vessel or vat for the production of a dairy product, such as a fermented milk product. Such cultures are in general referred to as “starter cultures” or “starters”.

In the present context, the term “packaged” relates to the final packaging of the product to obtain a product that can be supplied to a consumer. A suitable package may thus be a bag, e.g. a sealed plastic bag, a bottle, a container or similar, e.g. containing from 10 g to 5000 g, but it is presently preferred that a package contains from 50 g to 1000 g.

In the present context, the term “mutant” should be understood as a strain derived from a strain of the invention by means of e.g. genetic engineering, radiation and/or chemical treatment, and/or selection, adaptation, screening, etc. It is preferred that the mutant is a functionally equivalent mutant, e.g. a mutant that has substantially the same, or improved, properties (e.g. regarding yield, viscosity, gel stiffness, mouth coating, flavor, post acidification, acidification speed, and/or phage robustness) as the mother strain. Such a mutant is a part of the present invention. Especially, the term “mutant” refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood as one mutagenization step followed by a screening/selection step), but it is presently preferred that no more than 1000, no more than 100, no more than 20, no more than 10, or no more than 5, treatments are carried out. In a presently preferred mutant, less than 5%, or less than 1% or even less than 0.1% of the nucleotides in the bacterial genome have been shifted with another nucleotide, or deleted, compared to the mother strain.

In the present context, the term “variant” should be understood as a strain which is functionally equivalent to a strain of the invention, e.g. having substantially the same, or improved, properties e.g. regarding yield, viscosity, gel stiffness, mouth coating, flavor, post acidification, acidification speed, and/or phage robustness). Such variants, which may be identified using appropriate screening techniques, are a part of the present invention.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise dearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### EXAMPLES

### Example 1: Standard Cottage Cheese Manufacture

The skilled person knows how to produce cottage cheese and how it differentiates from other cheese (e.g. a soft cheese). A standard manufacture of cottage cheese providing a high cheese yield is described herein below.

Fermentation: Milk was fermented/coagulated 5-6 hours at 34° C. for short-set manufacture, whereby the fermentation time depended on the inoculation rate and the type of culture.

Cutting the curd: The pH was approximately 4.65 at the time the curd was cut (a pH>4.7 would have caused matting of the curd). The coagulum was cut by a frame cutter with 10 mm between the strings. First, the frame cutter was run 2 times vertically from side to side and then vertically from end to end followed by a horizontal run from end to end in the cheese vat until cubes of 10 mm were obtained.

The degree and type of agitation of curds after cutting and during cooking influences the end product greatly also in regards to yield:

Healing: During this time, 30 min., the curd was not agitated. The curd structure was fragile. The first agitation did not occur until 50 min. after starting the heating (cooking).

Cooking: The cooking was done step by step, at an approximate rate of 1° C. per 5 minutes.

The curd was cooked to expel the whey and to firm the individual curd particles. This was done slowly and with sufficient agitation to prevent the curds from matting, but gentle enough to prevent shattering of the curds.

This gave a uniform firming from surface to center in the curds throughout the vat and prevented matting and the formation of tough skin during cooking.

As mentioned above, the agitator was switched on 50 min after the start of cooking, 5 min later the temperature in the vat was measured (the temperature reached the target temperature of 38° C.).

The cooking and stirring process was continued until the temperature was 57° C. in the vat approximately 110-120 min. after cooking was started.

The whey was drained until the curd appeared and the jacket was drained.

Washing: The pH in the water for washing was adjusted to 5.0-5.2 with phosphoric acid and 3 ppm hypochlorite (0.18 ml/60 l) were added as well.

Wash 1. the jacket was drained and 60 l water at 12-13° C. were added to the vat (in this example=100 liter vat) and stirred for 15 min.—then the jacket was drained and filled with cold water.

Wash 2: 60 l water at 12-13° C. were added and stirred for 30 min, and then drained completely (trenching).

The drain was checked after trenching (10-20 min.) with a hand-held refractometer and a Brix of 1.0%±0.2 was found. (A Brix of greater than 1.2 could possibly indicate that not enough whey was removed on the initial drain prior to the first 60 liter wash.)

### Cream/Dressing for Cottage Cheese

The dressing was prepared the day before the production day.

A creaming ratio, i.e. curd to dressing of 60:40 was used. Other creaming ratios may also be employed.

A cream with 10.5% fat was made, wherein cream with 13-18% fat and skim milk was used. Optionally, 1-2% SMP may be added, and heated up to 50° C. and homogenized at 150 bars. Pasteurization at 90° C. for 20 min. and subsequent cooling to 10° C. may be carried out.

Further optionally NaCl (0.8-3.5%) may be added and the dressing may be stored at 3-5° C. till the next day.

### Mixing Curd/Dressing

The curd and dressing were weighed and mixed with each other very carefully in a plastic box (40×60 cm).

### Example 2: Novel strains of Lactococcus lactis subsp, Lactics and Lactococcus lactis subsp, Cremoris Increase Cheese Yield in a Cottage Cheese Make

Two novel strains of Lactococcus lactis subsp. lactis (DSM24650 and DSM24648) and two novel strains of Lactococcus lactis subsp. cremoris (DSM24649 and DSM21421) were tested in the standard procedure for making cottage cheese as described in example 1.

In order to compare the extra yield obtained by the use of these 4 mesophilic strains—the following setup was conducted:

A 50:50 combination of the Lactococcus lactic strain DSM21404 with the Streptococcus thermophilus strain DSM24653 was used as a reference blend.

### Reference Blend (Inoculation 0.030% in Skim Milk):

1A) 50% Lactococcus lactis DSM21404+50% Streptococcus thermophilus DSM24653

### Test Blends (Inoculation 0.030% In Skim Milk)—Focus on Extra Yield From Lactococcus Strains:

2) 50% Lactococcus lactis DSM24650+50% Lactococcus lactis DSM21404

3) 50% Lactococcus lactis DSM24548+50% Lactococcus lactis DSM21404

4) 50% Lactococcus lactis DSM 24649+50% Lactococcus lactis DSM21404

5) 50% Lactococcus lactis DSM21421+50% Lactococcus lactis DSM21404

This reference blend and the test blends were tested together in a Cottage Cheese manufacture that was completely identical:

After removing the whey and draining of the product, the total cheese weight was calculated. The ‘extra yield (%)’, i.e. the additional yield in comparison to the reference blend, was calculated in percent using the reference blend as a baseline. A summary of the ‘extra yield’ in percent for the novel strains of Lactococcus lactis can be seen in the Table below:

| Blend | Strain | Extra yield (%) |
|---|---|---|
| 1A Reference | DSM21404 | |
| 2 | DSM24649 | >6% |
| 3 | DSM24648 | >1% |
| 4 | DSM24650 | >9% |
| 5 | DSM21421 | >10% |

Based on these investigations it is clearly demonstrated that above Lactococcus strains have the ability to increase the cheese yield as tested in a cottage cheese make.

### Example 3: Streptococcus thermophilus Increasing Cheese Yield During the Manufacture of Cottage Cheese

Similar investigations were carried out for two strains of Streptococcus thermophilus (DSM24655 and DSM24654).

In order to compare the extra yield, i.e. the additional yield, obtained by using these 2 thermophilic Streptococcus thermophilus strains the following setup was conducted, using the same reference blend that was used in Example 2:

### Reference Blend Inoculation 0.030% in Skim Milk):

1B) 50% Lactococcus lactis DSM21404+50% Streptococcus thermophilus DSM24653

Test Blends (Inoculation 0.030% in Skim Milk)—Focus on Extra Yield From S. thermophilus Strains:

6) 50% Streptococcus thermophilus DSM24655+50% Streptococcus thermophilus DSM24653

7) 50% Streptococcus thermophilus DSM24654+50% Streptococcus thermophilus DSM24653

This reference blend and the test blends were tested together in a cottage cheese manufacture that was completely identical.

After removing the whey and draining of the product, the total cheese weight was calculated. The ‘extra yield (%)’, i.e. the additional yield in comparison to the reference blend, was calculated using the reference blend as a baseline. A summary of the ‘extra yield’ in percent for the novel strains of Streptococcus thermophilus can be seen in the Table below:

| Blend | Strain | Extra yield (%) |
|---|---|---|
| 1B Reference | DSM24653 | |
| 6 | DSM24655 | >6% |
| 7 | DSM24654 | >6% |
| 8 | ? | >2% |

Based on these investigations it is clearly demonstrated that above S. thermophilus strains have the ability to increase the cheese yield as tested in the standard cottage cheese manufacture.

### CONCLUSION

It is clearly demonstrated that some L. lactis, subsp lactis/subsp. cremoris and some S. thermophilus strains have a positive impact on cheese yield. In addition a significant quality difference was observed when comparing the cheese curd produced by the reference cultures as compared to the ‘extra yield’ cultures (examples 2+3):
1) Unexpectedly large cheese curds were observed when using the described ‘extra yield strains’ of Streptococcus thermophilus (ST) or Lactococcus lactis subspecies lactis/cremoris. In addition the curd was as firm as the reference and was very easy to handle.
2) In addition the curd demonstrated to have improved skin, which from a production point of view is very important for the curd handling.
3) As visualised on the FIG. 1 (cottage cheese manufactured with strain DSM24649) and 2 (cottage cheese manufactured with reference culture DSM21404) the curd had less fines, i.e. small particles. This means that the loss of fine curd particles during draining was minimised thereby increasing the yield.

The improvement of the described strains also had a clear impact on the final product, i.e. the curd plus dressing.
1) The overall mouth feel of the cottage cheese was significantly improved by using the investigated strains as compared to the reference culture.
2) Less residual whey was observed on the cottage cheese made with the novel strains.
3) When stirring in the final product—the reference blends were more fragile (curd broke down into fines) as compared to cottage cheese produced with Streptococcus thermophilus or Lactococcus lactis subspecies lactis/cremoris as described herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in ail possible variations thereof is encompassed by the invention unless otherwise indicated herein or otherwise clearly contradicted by context.

### DESCRIPTION OF THE FIGURES

The figures depict the impact of the novel strains on curd quality.

FIG. 1: Cottage cheese curd with extra yield manufactured with novel strain (DSM24649)

FIG. 2: Cottage cheese curd—manufactured with reference culture (DSM21404)

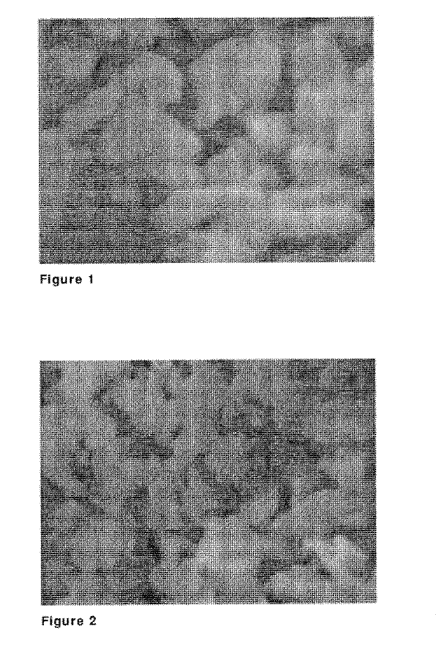

### DEPOSITS AND EXPERT SOLUTION

The following strains were deposited at the Leibniz institute DSMZ—German Collection of Microorganisms and Cell Cultures (formerly known as DSMZ—Deutsche Sammlung von Mikroorganismen und Zellkulturen GmbH), Inhoffenstr, 7B, D-38124 Braunschweig (DSM) on the mentioned dates, and given the mentioned accession numbers:

Novel Strains:

CHCC10812: Mar. 15, 2011, DSM24650

CHCC6086: Mar. 15, 2011, DSM24648

CHCC2618: Mar. 15, 2011, DSM24649

CHCC14308: Mar. 15, 2011, DSM24655

CHCC12828: Mar. 15, 2011, DSM24654

CHCC2907: Apr. 23, 2008, DSM21421

CHCC12942: Jul. 12, 2011, DSM 25012

Reference Strains:

CHCC12406: Mar. 15, 2011, DSM24653

CHCC1915: Apr. 23, 2008, DSM21404

The deposits were made according to the Budapest treaty on the international recognition of the deposit of microorganisms for the purposes of patent procedure.

The Applicant requests that a sample of the deposited microorganisms should be made available only to an expert approved by the Applicant.

### REFERENCES

U.S. Pat. No. 5,116,737

U.S. Pat. No. 6,962,721B1 (Texal, F R)

U.S. Pat. No. 3,298,836 (published 1967)

WO91/00690A1

“Gold Spot Dairy boost cottage cheese sales”, Dairy and Ice Cream Field, vol. 156, no. 6, 1973, pages 46-47.

R. Scott, (1986), Cheesemaking process, second ed., Elsevier Applied Science Publishers, London and New York.

G. Bylund, (1995), Dairy processing handbook, Tetra Pak Processing Systems, Lund, Sweden

F. Kosikowski, (1982), Cheese and fermented milk foods, second ed., Kosikowski & Associates, New York

All references cited in this patent document are hereby incorporated herein in their entirety by reference.

1-20. (canceled)

21. A method for manufacture of cottage cheese, which comprises the following steps:
a) inoculating a milk substrate with lactic acid bacteria belonging to a strain which is able to produce a capsular polysaccharide;
b) fermenting the milk with the bacteria; and
c) separating the cheese mass or curd from the whey.

22. The method of claim 21, wherein the strain is a strain of a Lactococcus species.

23. The method of claim 21, wherein the strain is a strain of a Streptococcus species.

24. The method of claim 21, wherein the strain is urease negative (Ur−).

25. The method of claim 21, wherein the strain is selected from the group consisting of strains that were deposited with the Leibniz Institute DSMZ—German Collection of Microorganisms and Cell Cultures under accession numbers: DSM24650, DSM24648, DSM24649, DSM24655, DSM24654, DSM21421, DSM 25012, and mutants and variants of any of these.

26. The method of claim 21, wherein the milk substrate is inoculated with 10⁴ to 10¹³ cfu/ml (cell forming units per milliliter), or more preferably inoculated with 10⁸ to 10¹² cfu/ml, of the bacteria.

27. The method of claim 21, which comprises a further step of adding a coagulant (such as rennet, rennin, a protease, and/or chymosin) before, during or after step a).

28. The method of claim 21, wherein the fermentation time in step (b) is from 3 to 10 hours.

29. The method of claim 21, wherein the milk substrate is milk, such as cow's milk.

30. The method of any of claim 21, wherein the step (c) includes one or more of the steps selected from the group consisting of:
i) cutting the coagulum at a pH between 4.6 and 4.7; and
ii) scalding (heating) of the curd after cutting.

31. The method of claim 21, wherein the resulting cheese curd is salted, pressed and packaged.

32. Cottage cheese, obtained by a method of claim 21.

33. The method of claim 21, wherein the strain belongs to the species Streptococcus thermophilus or Lactococcus lactis.

34. A lactic acid bacterial strain selected from the group consisting of strains that were deposited with the Leibniz Institute DSMZ—German Collection of Microorganisms and Cell Cultures under accession numbers: DSM24650, DSM24648, DSM24649, DSM24655, DSM24654, DSM 25012, and mutants derived from any of these strains, wherein said mutants have substantially the same, or improved, properties regarding cheese yield as the mother strain when used for the manufacture of cottage cheese.

35. The lactic acid bacterial strain of claim 34, wherein less than 1% (such as less than 0.1%) of the nucleotides in the bacterial genome of said mutants have been shifted with another nucleotide, or deleted, compared to the mother strain.
